(19) United States
(12) Patent Application Publication
(10) Pub. No.: US 2025/0231204 A1
(43) Pub. Date: Jul. 17, 2025

(54) EXHALED BIOMARKER FOR DIAGNOSIS AND PROGNOSIS OF PATIENT WITH IDIOPATHIC PULMONARY FIBROSIS

(71) Applicant: UNIVERSITY OF ULSAN FOUNDATION FOR INDUSTRY COOPERATION, Ulsan (KR)

(72) Inventor: Jin Woo SONG, Seoul (KR)

(73) Assignee: UNIVERSITY OF ULSAN FOUNDATION FOR INDUSTRY COOPERATION, Ulsan (KR)

(21) Appl. No.: 19/057,681

(22) Filed: Feb. 19, 2025

Related U.S. Application Data
(63) Continuation-in-part of application No. PCT/KR2023/012212, filed on Aug. 17, 2023.

(30) Foreign Application Priority Data
Aug. 19, 2022 (KR) 10-2022-0104343

(51) Int. Cl. G01N 33/92; A61K 31/4418; A61K 31/496; A61K 31/52; A61K 31/5377; A61K 31/573; A61K 31/675; A61P 11/00

(52) U.S. Cl. CPC G01N 33/92; A61K 31/4418; A61K 31/496; A61K 31/52; A61K 31/5377; A61K 31/573; A61K 31/675; A61P 11/00; G01N 2800/12; G01N 2800/52

(57) ABSTRACT

The present disclosure relates to an exhaled biomarker for the diagnosis and prognosis of patients with idiopathic pulmonary fibrosis. Among volatile organic compounds in respiratory gas, an exhaled biomarker was screened. The biomarker according to the present disclosure is a compound specific to idiopathic pulmonary fibrosis and not only has excellent performance in distinguishing idiopathic pulmonary fibrosis patients from normal control groups and other interstitial lung disease groups, but also allows for non-invasive diagnosis, and thus can be applied to elderly patients and advantageously used as an exhaled biomarker for the diagnosis and prognosis of patients with idiopathic pulmonary fibrosis.

### CROSS-REFERENCE TO RELATED APPLICATIONS

This application is a continuation-in-part of PCT Application PCT/KR2023/012212, filed on Aug. 17, 2023, and claims priority to Korean Patent Application No. 10-2022-0104343, filed on Aug. 19, 2022, the entire contents of each of which are incorporated herein by reference.

### TECHNICAL FIELD

The present disclosure relates to an exhaled biomarker for diagnosis and prognosis of patients with idiopathic pulmonary fibrosis.

### BACKGROUND ART

Idiopathic pulmonary fibrosis (IPF) is a fatal disease characterized by repeated inflammation in the lung interstitial tissue, leading to permanent scarring and tissue fibrosis. This results in structural changes in the lung tissue, ultimately causing impaired lung function and death. Approximately 5 million IPF patients occur worldwide, and in Korea, the incidence rate is 1.7 per 100,000 people. IPF accounts for more than 50% of interstitial lung diseases, making it the most commonly occurring condition among them.

IPF is a progressive disease that develops slowly over a period of one to two years, and as the disease progresses, it leads to the onset of respiratory difficulties. The average survival period is 60 months, but acute exacerbation occurs in 14% of patients annually. This disease shows low responsiveness to immunosuppressants and corticosteroids, and generally, treatment methods involve slowing the progression through pharmacological therapies such as antifibrotic agents.

Since the exact cause of IPF has not been identified, making treatment more challenging, it is crucial to detect the disease early through methods such as regular health check-ups. The diagnosis of IPF is made through typical chest CT imaging findings or surgical lung biopsy. However, in the case of chest imaging, there are often discrepancies in interpretation among readers, and in the case of surgical lung biopsy, it is often challenging to apply due to advanced age.

In addition to conventional diagnostic methods such as imaging, invasive testing, or biomarkers in body fluids such as blood, research on various diagnostic methods is being conducted to develop methods that can be selected even in cases where the aforementioned methods are difficult to apply. Exhaled biomarker diagnostic methods are one of them, and research on the discovery of specific substances in exhaled breath, materials, methods, or devices for adsorbing these substances has been conducted for various diseases. However, there is no known exhaled biomarker that shows significant effects in idiopathic pulmonary fibrosis or interstitial lung disease.

### DISCLOSURE

### Technical Problem

To solve the above problem, the present disclosure has developed the following information providing method and kit.

One object of the present disclosure is to provide a method for providing information for the diagnosis of idiopathic pulmonary fibrosis, comprising:
determining the level of one or more exhaled biomarkers selected from a group consisting of myristic acid, heptadecanoid acid, 5(S)-HETE (5-Hydroxyeicosatetraenoic acid), and 12(S)-HETE (12-Hydroxyeicosatetraenoic acid) collected from a subject.1

Another object of the present disclosure is to provide a diagnostic kit for idiopathic pulmonary fibrosis, comprising a composition for determining the level of the exhaled biomarkers and a specification describing the method for providing information as described above.

Another object of the present disclosure is to provide a method for providing information for the diagnosis of interstitial lung disease, comprising:
determining the level of one or more exhaled biomarkers selected from myristic acid or 5(S)-HETE collected from a subject.

Another object of the present disclosure is to provide a kit for diagnosing interstitial lung disease, comprising a composition for determining the level of the exhaled biomarker and a specification describing the method for providing information is described.

Another object of the present disclosure is to provide a method for detecting exhaled biomarkers and treating idiopathic pulmonary fibrosis, comprising:
(a) detecting a level of one or more exhaled biomarkers selected from the group consisting of myristic acid, heptadecanoic acid, 5(S)-HETE (5-Hydroxyeicosatetraenoic acid), and 12(S)-HETE (12-Hydroxyeicosatetraenoic acid) in exhaled breath collected from a subject; and
(b) treating the subject for idiopathic pulmonary fibrosis when the level of the one or more exhaled biomarker(s) selected from the group consisting of myristic acid, heptadecanoic acid, 5(S)-HETE, and 12(S)-HETE is increased compared to the corresponding exhaled biomarker level in a normal control group or an interstitial lung disease group;
wherein said treating comprises administering a therapeutically effective amount of a therapeutic agent.

Another object of the present disclosure is to provide a method for detecting exhaled biomarkers and treating interstitial lung disease, comprising:
(a) detecting a level of one or more exhaled biomarkers selected from the

group consisting of myristic acid and 5(S)-HETE (5-hydroxyeicosatetraenoic acid) in exhaled breath collected from a subject; and
(b) treating the subject for interstitial lung disease when the level of the one or more exhaled biomarkers selected from the group consisting of myristic acid and 5(S)-HETE is increased compared to the corresponding exhaled biomarker level in a normal control group;
wherein said treating comprises administering a therapeutically effective amount of a therapeutic agent.

However, the technical problems to be solved by the present disclosure are not limited to the problems mentioned above, and other unmentioned problems can be clearly understood by a person skilled in the art to which the present disclosure pertains from the description below.

### Technical Solution

To achieve the above objective, the present disclosure provides a method for providing information for the diagnosis of idiopathic pulmonary fibrosis, comprising:
determining the level of one or more exhaled biomarkers selected from a group consisting of myristic acid, heptadecanoid acid, 5(S)-HETE (5-Hydroxyeicosatetraenoic acid), and 12(S)-HETE (12-Hydroxyeicosatetraenoic acid) collected from a subject.

In one embodiment of the present disclosure, wherein the method further comprises comparing the level of one or more exhaled biomarkers selected from the group consisting of myristic acid, 5(S)-HETE, and 12(S)-HETE with the level of the same exhaled biomarker in a normal control group, but is not limited thereto.

In one embodiment of the present disclosure, wherein the method further comprises diagnosing idiopathic pulmonary fibrosis when the level of one or more exhaled biomarkers is increased compared to the normal control group, but is not limited thereto.

In one embodiment of the present disclosure, wherein the method further comprises comparing the level of one or more exhaled biomarkers selected from the group consisting of heptadecanoid acid or 5(S)-HETE with the level of the same exhaled biomarker in an interstitial lung disease group, but is not limited thereto.

In one embodiment of the present disclosure, wherein the method further comprises diagnosing idiopathic pulmonary fibrosis when the level of one or more exhaled biomarkers is increased compared to the interstitial lung disease group, but is not limited thereto.

In one embodiment of the present disclosure, wherein the method for providing information for the diagnosis of idiopathic pulmonary fibrosis has an AUC value of 0.63 or higher, but is not limited thereto.

Additionally, the present disclosure provides an idiopathic pulmonary fibrosis diagnostic kit comprising a composition for determining the exhaled biomarker, and a specification describing the information providing the method.

Additionally, the present disclosure provides a method for providing information for the diagnosis of interstitial lung disease, comprising:
determining the level of one or more exhaled biomarkers selected from myristic acid or 5(S)-HETE collected from a subject.

In one embodiment of the present disclosure, wherein the method further comprises comparing the level of one or more exhaled biomarkers with the level of the same exhaled biomarker in a normal control group, but is not limited thereto.

In one embodiment of the present disclosure, wherein the method further comprises diagnosing interstitial lung disease when the level of one or more exhaled biomarkers is increased compared to the normal control group, but is not limited thereto.

Additionally, the present disclosure provides an interstitial lung disease diagnostic kit comprising a composition for determining the exhaled biomarker, and a specification describing the information providing method.

Additionally, another object of the present disclosure is to provide a diagnostic use for idiopathic pulmonary fibrosis of the information providing method for the diagnosis of idiopathic pulmonary fibrosis, comprising the above steps.

Additionally, another object of the present disclosure is to provide a diagnostic use for interstitial pulmonary fibrosis of the information providing method for the diagnosis of interstitial pulmonary fibrosis, comprising the above steps.

Additionally, the present disclosure is a) administering a biological agent to a subject;
b) determining the level of one or more exhaled biomarker selected from a group consisting of myristic acid, heptadecanoid acid, 5(S)-HETE (5-Hydroxyeicosatetraenoic acid), and 12(S)-HETE (12-Hydroxyeicosatetraenoic acid) collected from the subject's exhaled breath;
c) comparing the level of the exhaled biomarker with the level of the same exhaled biomarker in a normal control group or an interstitial lung disease group; and
d) if the level of or more one of the exhaled biomarkers is decreased, re-administering the biological agent to the subject, a method for treating idiopathic pulmonary fibrosis.

The method of claim 12, wherein in step c),
when compared with a normal control group, the level of one or more exhaled biomarker selected from the group consisting of myristic acid, 5(S)-HETE, and 12(S)-HETE is compared with the level of the same exhaled biomarker in the normal control group; or
when compared with an interstitial lung disease group, the level of one or more exhaled biomarker selected from heptadecanoid acid or 5(S)-HETE is compared with the level of the same exhaled biomarker in the interstitial lung disease group, a method for treating idiopathic pulmonary fibrosis.

Additionally, the present disclosure is a) administering a biological agent to a subject;
b) determining the level of one or more exhaled biomarker selected from myristic acid or 5(S)-HETE (5-Hydroxyeicosatetraenoic acid) collected from the subject's exhaled breath;
c) comparing the level of the exhaled biomarker with the level of the same exhaled biomarker in a normal control group; and
d) if the level of one or more of the exhaled biomarkers is decreased, re-administering the biological agent to the subject, a method for treating interstitial lung disease.

Additionally, the present disclosure provides a use for a composition for diagnosing idiopathic pulmonary fibrosis or interstitial lung disease, comprising determining the level of one or more exhaled biomarker selected from a group consisting of myristic acid, heptadecanoid acid, 5(S)-HETE (5-Hydroxyeicosatetraenoic acid), and 12(S)-HETE (12-Hydroxyeicosatetraenoic acid).

Specifically, the present disclosure provides a use for a composition for diagnosing interstitial lung disease, comprising determining the level of one or more exhaled biomarker selected from myristic acid or 5(S)-HETE (5-Hydroxyeicosatetraenoic acid).

Additionally, the present disclosure provides a use for manufacturing a composition for diagnosing idiopathic pulmonary fibrosis or interstitial lung disease, comprising determining the level of one or more exhaled biomarker selected from a group consisting of myristic acid, heptadecanoid acid, 5(S)-HETE (5-Hydroxyeicosatetraenoic acid), and 12(S)-HETE (12-Hydroxyeicosatetraenoic acid).

Specifically, a use for producing a composition for diagnosing interstitial lung disease, comprising determining the level of one or more exhaled biomarker selected from myristic acid (myristic acid) or 5(S)-HETE (5-Hydroxyeicosatetraenoic acid), is provided.

Additionally, the present disclosure provides a diagnostic device for idiopathic pulmonary fibrosis or interstitial lung disease, comprising a composition that includes the exhaled biomarker-determining composition as an active ingredient.

To achieve the above objective, the present disclosure provides a method for detecting exhaled biomarkers and treating idiopathic pulmonary fibrosis, comprising:
(a) detecting a level of one or more exhaled biomarkers selected from the group consisting of myristic acid, heptadecanoic acid, 5(S)-HETE (5-Hydroxyeicosatetraenoic acid), and 12(S)-HETE (12-Hydroxyeicosatetraenoic acid) in exhaled breath collected from a subject; and
(b) treating the subject for idiopathic pulmonary fibrosis when the level of the one or more exhaled biomarker(s) selected from the group consisting of myristic acid, heptadecanoic acid, 5(S)-HETE, and 12(S)-HETE is increased compared to the corresponding exhaled biomarker level in a normal control group or an interstitial lung disease group;
wherein said treating comprises administering a therapeutically effective amount of a therapeutic agent.

In one embodiment of the present disclosure, wherein step (b), the subject:
is characterized by an increase in the level of one or more exhaled biomarkers, comprising myristic acid, 5(S)-HETE, or 12(S)-HETE, when compared to the corresponding level in a normal control group; or
is characterized by an increase in the level of one or more exhaled biomarkers, comprising heptadecanoic acid or 5(S)-HETE, when compared to the corresponding level in an interstitial lung disease (ILD) group, but is not limited thereto.

In one embodiment of the present disclosure, wherein the exhaled biomarkers comprise one or more free fatty acids selected from the group consisting of myristic acid, pentadecanoic acid, palmitic acid, heptadecanoic acid, and stearic acid, and one or more arachidonic acid metabolites selected from the group consisting of LTB4, 5(S)-HETE, 12(S)-HETE, 17(S)-DiHDoHE3, 11,12-EET, and 8(9)-DHET,
wherein myristic acid, heptadecanoic acid, 5(S)-HETE, and 12(S)-HETE are selected based on the following criteria, but is not limited thereto:
(i) when the control group is a normal control group, the p-value obtained from a t-test comparing the content of exhaled breath condensate between the subject and the normal control group is 0.019 or less for free fatty acids, and 0.006 or less for arachidonic acid metabolites; or
(ii) when the control group is an interstitial lung disease group, the p-value obtained from a t-test comparing the content of exhaled breath condensate between the subject and the interstitial lung disease group is 0.048 or less for free fatty acids, and less than 0.019 for arachidonic acid metabolites.

In one embodiment of the present disclosure, wherein the exhaled sample is exhaled breath condensate (EBC), but is not limited thereto.

In one embodiment of the present disclosure, wherein, in the step (b), when compared to a normal control group, the AUC (area under the ROC curve) value for myristic acid, 5(S)-HETE, and 12(S)-HETE is greater than 0.63, or the p-value associated therewith is less than or equal to 0.049, but is not limited thereto.

In one embodiment of the present disclosure, wherein, in the step (b), when compared to an interstitial lung disease group, the AUC value for heptadecanoic acid and 5(S)-HETE is greater than 0.64, or the p-value associated therewith is less than or equal to 0.04, but is not limited thereto.

To achieve the above objective, the present disclosure provides a method for detecting exhaled biomarkers and treating interstitial lung disease, comprising:
(a) detecting a level of one or more exhaled biomarkers selected from the group consisting of myristic acid and 5(S)-HETE (5-hydroxyeicosatetraenoic acid) in exhaled breath collected from a subject; and
(b) treating the subject for interstitial lung disease when the level of the one or more exhaled biomarkers selected from the group consisting of myristic acid and 5(S)-HETE is increased compared to the corresponding exhaled biomarker level in a normal control group;
wherein said treating comprises administering a therapeutically effective amount of a therapeutic agent.

In one embodiment of the present disclosure, wherein, the AUC value for myristic acid and 5(S)-HETE is greater than 0.6, or the p-value associated therewith is less than or equal to 0.026, but is not limited thereto.

### Advantageous Effects

According to the exhaled biomarker for the diagnosis and prognosis of patient with idiopathic pulmonary fibrosis, an exhaled biomarker was selected from the volatile organic compounds in exhaled gases. The biomarker of the present disclosure is a compound specific to idiopathic pulmonary fibrosis, which not only shows excellent performance in distinguishing the idiopathic pulmonary fibrosis patient group from the normal control and interstitial lung disease groups but also enables diagnosis through a non-invasive method. Therefore, it can be effectively utilized as an exhaled biomarker for the diagnosis and prognosis of idiopathic pulmonary fibrosis, which can also be applied to elderly patients.

### BRIEF DESCRIPTION OF THE DRAWINGS

FIG. 1 shows the process of obtaining exhaled gas and exhaled condensate.

FIG. 2 is a schematic diagram showing an analytical platform targeting specific metabolic pathways.

FIG. 3 shows a curve graph of ROC analysis results demonstrating the diagnostic ability of exhaled biomarkers between the idiopathic pulmonary fibrosis patient group and the normal control group.

FIG. 4 shows a ROC curve graph demonstrating the diagnostic ability of exhaled biomarkers between the idiopathic pulmonary fibrosis patient group and the disease control group.

FIG. 5 shows a ROC curve graph demonstrating the diagnostic ability of exhaled biomarkers between the disease control group and the normal control group.

### BEST MODE

The inventors have identified a biomarker in the exhalation of a subject that can diagnose idiopathic pulmonary fibrosis in one embodiment of the present disclosure, thereby completing the present disclosure.

The present disclosure provides a method for providing information for the diagnosis of idiopathic pulmonary fibrosis, comprising:
determining the level of one or more exhaled biomarkers selected from a group consisting of myristic acid, heptadecanoid acid, 5(S)-HETE (5-Hydroxyeicosatetraenoic acid), and 12(S)-HETE (12-Hydroxyeicosatetraenoic acid) collected from a subject.

The results of the analysis of the exhaled breath condensate indicate that the metabolites in the exhaled breath are free fatty acids, which may include, for example, myristic acid (C14:0), pentadecanoic acid (C15:0), palmitic acid (C16:0), heptadecanoic acid (C17:0), and stearic acid (18:0), but are not limited thereto. Additionally, the metabolites in the exhaled breath may be eicosanoids, such as, for example, LTB4 (Leukotriene B4), 5(S)-HETE (5-Hydroxyeicosatetraenoic acid), 12(S)-HETE (12-Hydroxyeicosatetraenoic acid), 10(S), 17(S)-DiHDoHE, 11,12-EET (11,12-Epoxyeicosatrienoic acid), and 8(9)-DHET (8, 9-Dihydroxyeicosatrienoic acid).

In one embodiment of the present disclosure, the method for providing information for the diagnosis of idiopathic pulmonary fibrosis may further include comparing the level of one or more exhaled biomarkers selected from the group consisting of myristic acid, 5(S)-HETE, and 12(S)-HETE with the level of the same exhaled biomarkers in a normal control group, but is not limited thereto.

In one embodiment of the present disclosure, the method may further include diagnosing idiopathic pulmonary fibrosis when the level of one or more of the exhaled biomarkers is increased compared to the normal control group, but is not limited thereto.

In one embodiment of the present disclosure, the method for providing information for the diagnosis of idiopathic pulmonary fibrosis may further include comparing the level of one or more exhaled biomarkers selected from heptadecanoic acid or 5(S)-HETE with the level of the same exhaled biomarker in the interstitial lung disease group, but is not limited thereto.

In one embodiment of the present disclosure, the method for providing information for the diagnosis of idiopathic pulmonary fibrosis may further include diagnosing idiopathic pulmonary fibrosis when the level of one or more exhaled biomarkers is increased compared to the interstitial lung disease group, but is not limited thereto.

In the present disclosure, “exhalation” refers to the breathing in the outward direction from an organism, meaning the air that moves from the lungs to the airways, also known as exhaled breath.

A commonly used exhalation collection device may be used to collect the exhalation of the subject, such as a high-concentration exhalation collection device or an in vitro diagnostic device equipped with sensors.

The exhalation collection device may further include auxiliary devices such as a mouthpiece to facilitate exhalation by the subject, and additional devices may be included to prevent the collection of unnecessary saliva, foreign substances, moisture, and the like. Additionally, a condenser may be included to convert the exhaled compounds into a state that is easy to detect, but is not limited thereto.

The collected exhalation may undergo a phase change to facilitate the detection of exhaled biomarkers, and depending on the collection device, it can be collected in a state of matter corresponding to gas, liquid, or solid.

The exhalation may undergo a phase change to a liquid state through conventional processes such as cooling or pressurization, and the liquefied exhalation may be in the form of a condensate, but is not limited thereto. Additionally, the exhalation may be solidified and obtained in a solid form, or it may be maintained in a gaseous state and obtained as such.

According to an embodiment of the present disclosure, the exhaled air collected from the subject may come into contact with the cooled surface of a condenser and be used in the form of exhaled breath condensate.

In the present disclosure, the “exhaled breath condensate” may be pretreated to facilitate the detection of biomarkers in the exhaled air. For example, homogenization, filtration, distillation, extraction, or concentration processes may be applied for temperature control, humidity control, and removal of unnecessary foreign substances, and processes for inactivating interfering components may be applied, with reagents or the like being added for this purpose.

The gas state, cooled liquid, or solidified exhaled air may be analyzed immediately or stored for a certain period before analysis, and if necessary, substances required for storage may be added, but is not limited thereto.

The exhaled breath condensate can be classified as a volatile or non-volatile polymer substance.

The method for discovering biomarkers specific to idiopathic pulmonary fibrosis or interstitial lung disease in exhaled breath condensate may include techniques such as GC-MS (Gas Chromatography-Mass Spectrometry), LC-MS/MS [MRM] (Liquid Chromatography-Mass Spectrometry with multiple reaction monitoring), or SPME (Solid Phase Micro-Extraction), but is not limited thereto.

According to one embodiment of the present disclosure, after concentrating the substances to be analyzed for free fatty acids in exhaled air, the SPME technique may be applied. Additionally, a fiber coated with a material capable of adsorbing organic compounds may be placed in a container containing the exhaled gas to adsorb the organic compounds. The fiber is then placed in the injector of a GC-MS, where the organic compounds are desorbed at a high temperature, allowing the compounds to enter the analytical device for analysis.

The analysis of exhaled breath condensate may be conducted using the same methods as metabolite analysis in conventional blood samples. For example, free fatty acids in exhaled breath condensate may be extracted using an organic solvent, then derivatized through a chemical reaction, followed by the application of GC-MS, but is not limited thereto.

The method for providing information for the diagnosis of idiopathic pulmonary fibrosis using one or more exhaled biomarkers selected from the group consisting of myristic acid, 5(S)-HETE, and 12(S)-HETE, as compared to the normal control group, may have an AUC value measured between 0.635 and 0.756, with P-values measured at 0.003, <0.001, and 0.049, respectively.

The method for providing information for the diagnosis of idiopathic pulmonary fibrosis using one or more exhaled biomarkers selected from the group consisting of heptadecanoic acid or 5(S)-HETE, as compared to the interstitial lung disease group, may have an AUC value measured between 0.642 and 0.699, with P-values measured at 0.04 and 0.004, respectively.

In one embodiment of the present disclosure, the method for providing information for the diagnosis of idiopathic pulmonary fibrosis may have an AUC value of 0.63 or higher.

In one embodiment of the present disclosure, to diagnose idiopathic pulmonary fibrosis (IPF), exhaled breath condensate (EBC) was analyzed using GC-MS, SPME, and LC-MS/MS. The analysis revealed the presence of five free fatty acids and six arachidonic acid metabolites in 1 mL of EBC per subject.

Among the five free fatty acids and six arachidonic acid metabolites, the following results were obtained: When compared to the normal control group, the levels of myristic acid among the free fatty acids, and 5(S)-HETE and 12(S)-HETE among the arachidonic acid metabolites were found to be elevated in the EBC of the IPF group. The p-values for these three substances, as determined by a t-test between the two groups, were 0.019, <0.001, and 0.006, respectively. Therefore, the p-value for distinguishing the IPF group from the normal control group based on the free fatty acids and arachidonic acid metabolites in the EBC is ≤0.019 for the free fatty acids and ≤0.006 for the arachidonic acid metabolites, with a combined value ≤0.019. Consequently, the other four free fatty acids and four arachidonic acid metabolites were excluded.

Secondly, when compared to the interstitial lung disease (ILD) group, higher concentrations of heptadecanoic acid among the free fatty acids and 5(S)-HETE among the arachidonic acid metabolites were observed in the EBC of the IPF group. The p-values for these two substances, as determined by a t-test between the two groups, were 0.048 and 0.004, respectively. Therefore, the p-value for distinguishing the IPF group from the ILD group based on the free fatty acids and arachidonic acid metabolites in the EBC is ≤0.048 for the free fatty acids and <0.019 for the arachidonic acid metabolites, with a combined value ≤0.048. As a result, the other four free fatty acids and five arachidonic acid metabolites were excluded.

Additionally, the present disclosure provides a diagnostic kit for idiopathic pulmonary fibrosis, comprising a composition for determining the exhaled biomarker and a specification describing the information providing method.

In the present disclosure, the composition for determining the exhaled biomarker may include proteins, polynucleotides, nucleic acids, compounds, antibodies, aptamers, or the like that specifically bind to the marker substances, but is not limited thereto. In general, any formulation that can be used to confirm the exhaled biomarker may be applied.

In the present disclosure, the term “biomarker” refers to a marker that can distinguish between normal and pathological states, predict treatment responses, and be objectively measured. “Idiopathic pulmonary fibrosis biomarker” or “interstitial lung disease biomarker” refers to cells, proteins, DNA, RNA, metabolites, etc., that can be used to distinguish and diagnose patients with idiopathic pulmonary fibrosis or interstitial lung disease from the exhaled breath of a subject. In the present disclosure, biomarkers are provided for distinguishing patients with idiopathic pulmonary fibrosis from a normal control group or an interstitial lung disease group, as well as biomarkers for distinguishing patients with interstitial lung disease from a normal control group.

In the present disclosure, the term “protein” is used interchangeably with “polypeptide” or “peptide,” and refers to a polymer of amino acid residues, as conventionally found in naturally occurring proteins.

In the present disclosure, the term “polynucleotide” or “nucleic acid” refers to deoxyribonucleic acid (DNA) or ribonucleic acid (RNA) in the form of single-stranded or double-stranded molecules. Unless otherwise limited, the term “nucleic acid” also includes known analogs of naturally occurring nucleotides that hybridize with nucleic acids in a manner similar to naturally occurring nucleotides.

In the present disclosure, the term “antibody” refers to a specific protein molecule directed against an antigenic site. For the purposes of the present disclosure, the term “antibody” refers to an antibody that specifically binds to a marker protein, and includes polyclonal antibodies, monoclonal antibodies, and recombinant antibodies.Additionally, any portion of an antibody that retains antigen-antibody binding ability is included as an antibody of the present disclosure, and all types of immunoglobulin antibodies that specifically bind to the exhaled biomarkers presented in the present disclosure are included. For example, the complete form of an antibody having two full-length light chains and two full-length heavy chains, as well as functional fragments of the antibody molecule, including Fab, F(ab′), F(ab′)2, and Fv, which possess antigen-binding functionality, are included. Furthermore, the antibodies of the present disclosure include special antibodies such as humanized antibodies, chimeric antibodies, and recombinant antibodies, as long as they can specifically bind to the proteins of the present disclosure.

In the present disclosure, the term “aptamer” refers to a single-stranded nucleic acid (DNA, RNA, or modified nucleic acid) that has a stable tertiary per se structure and can specifically bind to an analyte to be detected in a sample, thereby enabling the specific detection of the presence of a target protein in the sample.

The preparation of the aptamer can be performed according to conventional methods for aptamer production, which involves determining and synthesizing the sequence of oligonucleotides with selective and high binding affinity for the target protein to be detected. Subsequently, the 5′ or 3′ end of the oligonucleotide can be modified with —SH, —COOH, —OH, or NH2 groups to enable binding to functional groups on the aptamer chip, but is not limited thereto.

The composition for detecting the exhaled biomarker may include salts, compounds, and substances that serve additional functional roles, which can all be used generally in the preparation. These substances may be present separately for addition when using the kit, but are not limited thereto.

Additionally, the present disclosure provides a method for providing information for the diagnosis of interstitial lung disease, comprising:
determining the level of one or more exhaled biomarkers selected from myristic acid or 5(S)-HETE collected from a subject.

In one embodiment of the present disclosure, the method for providing information for the diagnosis of interstitial lung disease may further include comparing the level of one or more of the exhaled biomarkers with the level of the same biomarker in a normal control group.

In one embodiment of the present disclosure, the method may further include diagnosing interstitial lung disease if the level of one or more of the exhaled biomarkers is increased compared to the normal control group, but is not limited thereto.

The method for providing information for the diagnosis of interstitial lung disease using one or more exhaled biomarkers selected from myristic acid or 5(S)-HETE, as compared to the normal control group, may have an AUC value measured between 0.642 and 0.680, with P-values of 0.026 and 0.005, respectively.

In one embodiment of the present disclosure, the method for providing information for the diagnosis of interstitial lung disease may have an AUC value of 0.642 or higher.

According to the present disclosure, when there are two or more exhaled biomarkers of the present disclosure for diagnosing idiopathic fibrosis patients from a disease control group or a normal control group, at least one of the two or more markers can be detected at a higher level compared to each comparison group, allowing diagnosis of the idiopathic fibrosis patient group. The same applies for diagnosing the interstitial lung disease group.

In this case, the marker levels in the sample may be used as the basis for comparison with each comparison group, but is not limited thereto. Quantitative values identified in an embodiment of the present disclosure, such as the cutoff level

Additionally, the present disclosure provides a kit for diagnosing interstitial lung disease, which includes a formulation for detecting the exhaled biomarkers and a specification describing the information providing method.

The five free fatty acids and six arachidonic acid metabolites according to the present disclosure may be associated with lung function.

Lung function may be evaluated by FVC (forced vital capacity), FEV1 (forced expiratory volume in 1 second), DLCO (Diffusing capacity of the Lung for Carbon monocide (CO)), and TLC (total lung capacity), but is not limited thereto.

FVC refers to the forced vital capacity, which is the volume of air exhaled after taking a maximal inhalation followed by a maximal effort of exhalation.

FEV1 refers to the forced expiratory volume in 1 second, which is an indicator of how quickly a person can exhale air during the first second of a forced exhalation.

DLCO is measured by having the subject inhale a small amount of carbon monoxide gas, hold their breath for 10 seconds, and then exhale. This test provides an indication of how efficiently gas exchange occurs in the lungs.

TLC refers to the total volume of the lungs, which is the sum of the maximum amount of air inhaled, the maximum amount of air exhaled, and the residual volume.

The lung function according to the present disclosure may have a significant correlation with lung diffusing capacity, and among the arachidonic acid metabolites, 11,12-EET may be associated with total lung capacity.

Furthermore, in one embodiment of the present disclosure, the five free fatty acids and six arachidonic acid metabolites were found to have a statistically significant correlation with one or more pulmonary function parameters selected from FVC, FEV1, DLCO, and TLC. These pulmonary function parameters may serve as biomarkers for the treatment of IPF and interstitial lung disease in patients with impaired lung function.

In the present disclosure, “confirmation” or “determination” may include quantifying the concentration of a detected or measured target, such as “detection” or “measurement,” and it also includes qualitative confirmation of the presence or absence of a specific substance. Therefore, it encompasses both measuring and confirming (determining) the presence (expression) of the target substance and measuring and confirming changes in the level (expression level) of the target substance.

In the present disclosure, the term “diagnosis” refers to determining the susceptibility of a subject to a specific disease or condition, determining whether the subject currently has a specific disease or condition, assessing the prognosis of a subject with a specific disease or condition, or including therametrics (e.g., monitoring the status of an object to provide information about therapeutic efficacy).

In the present invention, “prognostic prediction” or “prognosis” refers to the prediction of the disease progression in a patient population with the disease of the present invention. It can refer to predicting the probabilities of disease progression, worsening, relapse, or stability, etc., through the increase or decrease in the levels of the biomarkers of the present invention.

In the present disclosure, the term “kit” refers to a tool or device that includes tools for distinguishing an idiopathic pulmonary fibrosis patient from a normal control group or an interstitial lung disease group, or for distinguishing an interstitial lung disease patient from a normal control group.

The kit of the present disclosure may include a composition capable of detecting the exhaled biomarkers according to the present disclosure, as well as other components, compositions, solutions, devices, and the like conventionally required for detecting these biomarkers. There are no specific restrictions regarding the order of applying the aforementioned materials, and the application of each material may proceed simultaneously or sequentially.

In the present disclosure, the kit may further include a container or the like but is not limited thereto. The container may serve the role of packaging the substances, as well as the role of storing and securing them. The material of the container may be, for example, plastic, glass bottles, etc., but is not limited thereto.

In the present disclosure, the term “analysis” may preferably mean “measurement,” and the qualitative analysis may refer to measuring and confirming (determining) the presence of the intended substance, while the quantitative analysis may refer to measuring and confirming (determining) the change in the presence level (expression level) or amount of the intended substance.

In the present disclosure, analysis or measurement may be performed without limitation, including both qualitative and quantitative methods, and quantitative measurement may be performed.

Additionally, the present disclosure provides a method for treating idiopathic pulmonary fibrosis, comprising: detecting the biomarker in the subject's exhaled air using a composition for detecting the exhaled biomarker of the present disclosure; comparing the exhaled biomarker with a normal control group or an interstitial lung disease group; and treating idiopathic pulmonary fibrosis.

Additionally, the present disclosure provides a method for treating interstitial lung disease, comprising: detecting the biomarker in the subject's exhaled air using a composition for detecting the exhaled biomarker of the present disclosure; comparing the exhaled biomarker level with a normal control group; and treating interstitial lung disease.

Additionally, the present disclosure is a) administering a biological agent to a subject;
b) determining the level of one or more exhaled biomarker selected from a group consisting of myristic acid, heptadecanoid acid, 5(S)-HETE (5-Hydroxyeicosatetraenoic acid), and 12(S)-HETE (12-Hydroxyeicosatetraenoic acid) collected from the subject's exhaled breath;
c) comparing the level of the exhaled biomarker with the level of the same exhaled biomarker in a normal control group or an interstitial lung disease group; and
d) if the level of one or more of the exhaled biomarkers is decreased, re-administering the biological agent to the subject, a method for treating idiopathic pulmonary fibrosis.

The method of claim 12, wherein in step c),
when compared with a normal control group, the level of one or more exhaled biomarker selected from the group consisting of myristic acid, 5(S)-HETE, and 12(S)-HETE is compared with the level of the same exhaled biomarker in the normal control group; or
when compared with an interstitial lung disease group, the level of one ore more exhaled biomarker selected from heptadecanoid acid or 5(S)-HETE is compared with the level of the same exhaled biomarker in the interstitial lung disease group, a method for treating idiopathic pulmonary fibrosis.

Additionally, the present disclosure is a) administering a biological agent to a subject;
b) determining the level of one or more exhaled biomarker selected from myristic acid or 5(S)-HETE (5-Hydroxyeicosatetraenoic acid) collected from the subject's exhaled breath;
c) comparing the level of the exhaled biomarker with the level of the same exhaled biomarker in a normal control group; and
d) if the level of one or more of the exhaled biomarkers is decreased, re-administering the biological agent to the subject, a method for treating interstitial lung disease.

In the present disclosure, the “method for treating idiopathic pulmonary fibrosis” or “method for treating interstitial lung disease” may be applied simultaneously or sequentially with general treatment methods for treating idiopathic pulmonary fibrosis or interstitial lung disease, but is not limited thereto.

In the present disclosure, the “method for treating idiopathic pulmonary fibrosis” or “method for treating interstitial lung disease” may involve the co-prescription of a prophylactic or therapeutic composition for preventing or treating idiopathic pulmonary fibrosis or interstitial lung disease.

To achieve the above objective, the present disclosure provides a method for detecting exhaled biomarkers and treating idiopathic pulmonary fibrosis, comprising:
(a) detecting a level of one or more exhaled biomarkers selected from the group consisting of myristic acid, heptadecanoic acid, 5(S)-HETE (5-Hydroxyeicosatetraenoic acid), and 12(S)-HETE (12-Hydroxyeicosatetraenoic acid) in exhaled breath collected from a subject; and
(b) treating the subject for idiopathic pulmonary fibrosis when the level of the one or more exhaled biomarker(s) selected from the group consisting of myristic acid, heptadecanoic acid, 5(S)-HETE, and 12(S)-HETE is increased compared to the corresponding exhaled biomarker level in a normal control group or an interstitial lung disease group;
wherein said treating comprises administering a therapeutically effective amount of a therapeutic agent.

In one embodiment of the present disclosure, wherein step (b), the subject:
is characterized by an increase in the level of one or more exhaled biomarkers, comprising myristic acid, 5(S)-HETE, or 12(S)-HETE, when compared to the corresponding level in a normal control group; or
is characterized by an increase in the level of one or more exhaled biomarkers, comprising heptadecanoic acid or 5(S)-HETE, when compared to the corresponding level in an interstitial lung disease (ILD) group, but is not limited thereto.

In one embodiment of the present disclosure, wherein the exhaled biomarkers comprise one or more free fatty acids selected from the group consisting of myristic acid, pentadecanoic acid, palmitic acid, heptadecanoic acid, and stearic acid, and one or more arachidonic acid metabolites selected from the group consisting of LTB4, 5(S)-HETE, 12(S)-HETE, 17(S)-DiHDoHE3, 11,12-EET, and 8(9)-DHET,
wherein myristic acid, heptadecanoic acid, 5(S)-HETE, and 12(S)-HETE are selected based on the following criteria, but is not limited thereto:
(i) when the control group is a normal control group, the p-value obtained from a t-test comparing the content of exhaled breath condensate between the subject and the normal control group is 0.019 or less for free fatty acids, and 0.006 or less for arachidonic acid metabolites; or
(ii) when the control group is an interstitial lung disease group, the p-value obtained from a t-test comparing the content of exhaled breath condensate between the subject and the interstitial lung disease group is 0.048 or less for free fatty acids, and less than 0.019 for arachidonic acid metabolites.

In one embodiment of the present disclosure, wherein the exhaled sample is exhaled breath condensate (EBC), but is not limited thereto.

In one embodiment of the present disclosure, wherein, in the step (b), when compared to a normal control group, the AUC (area under the ROC curve) value for myristic acid, 5(S)-HETE, and 12(S)-HETE is greater than 0.63, or the p-value associated therewith is less than or equal to 0.049, but is not limited thereto.

In one embodiment of the present disclosure, in step (b), when compared to a normal control group, the AUC (area under the ROC curve) values of the myristic acid, the 5(S)-HETE, and the 12(S)-HETE may be greater than 0.63, and may specifically be greater than 0.631, 0.632, 0.633, 0.634, or 0.635 or more, but are not limited thereto.

In one embodiment of the present disclosure, wherein, in the step (b), when compared to an interstitial lung disease group, the AUC value for heptadecanoic acid and 5(S)-HETE is greater than 0.64, or the p-value associated therewith is less than or equal to 0.04, but is not limited thereto.

In one embodiment of the present disclosure, in step (b), when compared to an interstitial lung disease group, the AUC values of the heptadecanoic acid and the 5(S)-HETE may be greater than 0.64, and may specifically be greater than 0.641 or 0.642 or more, but are not limited thereto.

To achieve the above objective, the present disclosure provides a method for detecting exhaled biomarkers and treating interstitial lung disease, comprising:
(a) detecting a level of one or more exhaled biomarkers selected from the group consisting of myristic acid and 5(S)-HETE (5-hydroxyeicosatetraenoic acid) in exhaled breath collected from a subject; and
(b) treating the subject for interstitial lung disease when the level of the one or more exhaled biomarkers selected from the group consisting of myristic acid and 5(S)-HETE is increased compared to the corresponding exhaled biomarker level in a normal control group;
wherein said treating comprises administering a therapeutically effective amount of a therapeutic agent.

In one embodiment of the present disclosure, wherein, the AUC value for myristic acid and 5(S)-HETE is greater than 0.6, or the p-value associated therewith is less than or equal to 0.026, but is not limited thereto.

In one embodiment of the present disclosure, the AUC values of myristic acid and 5(S)-HETE may be greater than 0.6, 0.61, 0.62, 0.63, 0.64, 0.641, or 0.642 or more, but are not limited thereto.

The pharmaceutical composition for prevention or treatment of the present disclosure may further include an appropriate carrier, excipient, and diluent commonly used in the preparation of pharmaceutical compositions. The excipient, for example, may be one or more selected from the group consisting of a diluent, binder, disintegrant, lubricant, adsorbent, humectant, film-coating material, and controlled-release additive.

The pharmaceutical composition of the present disclosure can be formulated and used in various forms, including powders, granules, sustained-release granules, enteric-coated granules, liquids, eye drops, elixirs, emulsions, suspensions, tinctures, troches, aromatic waters, limonades, tablets, sustained-release tablets, enteric-coated tablets, sublingual tablets, hard capsules, soft capsules, sustained-release capsules, enteric-coated capsules, lozenges, tinctures, soft extracts, dry extracts, liquid extracts, injections, capsules, perfusates, suppositories, lotions, pastes, sprays, inhalants, patches, sterilized injectable solutions, or aerosols, and the external preparations may include forms such as creams, gels, patches, sprays, ointments, lotions, liniments, pastes, or cataplasms.

The carriers, excipients, and diluents that can be included in the pharmaceutical composition of the present disclosure include lactose, dextrose, sucrose, oligosaccharides, sorbitol, mannitol, xylitol, erythritol, maltitol, starch, acacia gum, alginate, gelatin, calcium phosphate, calcium silicate, cellulose, methylcellulose, amorphous cellulose, polyvinylpyrrolidone, water, methylhydroxybenzoate, propylhydroxybenzoate, talc, magnesium stearate, and mineral oil.

When formulating the preparation, diluents or excipients such as commonly used fillers, extenders, binders, wetting agents, disintegrants, surfactants, and similar substances are used.

The excipients that may be used in the tablets, powders, granules, capsules, pills, and troches of the present disclosure include excipients such as corn starch, potato starch, wheat starch, lactose, sucrose, glucose, fructose, D-mannitol, precipitated calcium carbonate, synthetic aluminum silicate, calcium dihydrogen phosphate, calcium sulfate, sodium chloride, sodium bicarbonate, purified lanolin, microcrystalline cellulose, dextrin, sodium alginate, methylcellulose, sodium carboxymethylcellulose, kaolin, urea, colloidal silica gel, hydroxypropyl starch, hydroxypropylmethylcellulose (HPMC), HPMC 1828, HPMC 2906, HPMC 2910, propylene glycol, casein, calcium lactate, Primojel, etc. as diluents; gelatin, gum arabic, ethanol, agar powder, cellulose acetate phthalate, carboxymethylcellulose, calcium carboxymethylcellulose, glucose, purified water, sodium caseinate, glycerin, stearic acid, sodium carboxymethylcellulose, methylcellulose sodium, methylcellulose, microcrystalline cellulose, dextrin, hydroxycellulose, hydroxypropyl starch, hydroxy-methylcellulose, purified shellac, starch powder, hydroxypropyl cellulose, hydroxypropylmethylcellulose, polyvinyl alcohol, polyvinylpyrrolidone, etc. as binders; hydroxypropylmethylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, anhydrous silica, 1-hydroxypropyl cellulose, dextran, ion exchange resin, polyvinyl acetate, formaldehyde-treated casein and gelatin, alginate, amylose, guar gum, sodium bicarbonate, polyvinylpyrrolidone, calcium phosphate, gel-forming starch, gum arabic, amylopectin, pectin, sodium polyphosphate, ethylcellulose, sucrose, magnesium aluminum silicate, D-sorbitol solution, and anhydrous silica as disintegrants; calcium stearate, magnesium stearate, stearic acid, hydrogenated vegetable oil, talc, kaolin, baselin, sodium stearate, cocoa butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, macrogol, synthetic aluminum silicate, anhydrous silica, long-chain fatty acids, high alcohols, silicone oil, paraffin oil, polyethylene glycol fatty acid esters, starch, sodium chloride, sodium acetate, sodium oleate, dl-leucine, and anhydrous silica as lubricants.

The excipients that may be used in the liquid preparations of the present disclosure include water, dilute hydrochloric acid, dilute sulfuric acid, sodium citrate, mono-stearate sucrose, polyoxyethylene sorbitan fatty acid esters (Tween esters), polyoxyethylene monoalkyl ethers, lanolin ethers, lanolin esters, acetic acid, hydrochloric acid, ammonia solution, ammonium bicarbonate, potassium hydroxide, sodium hydroxide, prolamine, polyvinylpyrrolidone, ethylcellulose, sodium carboxymethylcellulose, and similar substances.

The syrup of the present disclosure may be used in the solution of sucrose, other sugars, or sweeteners, and similar substances, and may be used, as needed, flavoring agents, coloring agents, preservatives, stabilizers, suspending agents, emulsifiers, thickeners, and similar substances.

The emulsion of the present disclosure may be used in purified water, and may be used, as needed, emulsifiers, preservatives, stabilizers, flavoring agents, and similar substances.

The suspension of the present disclosure may be used in suspending agents such as acacia, tragacanth, methylcellulose, carboxymethylcellulose, carboxymethylcellulose sodium, microcrystalline cellulose, sodium alginate, hydroxypropylmethylcellulose (HPMC), HPMC 1828, HPMC 2906, HPMC 2910, and similar substances, and may be used, as needed, surfactants, preservatives, stabilizers, colorants, flavoring agents, and similar substances.

The injectable formulations of the present disclosure may include solvents such as purified water for injection, 0.9% sodium chloride injection, Ringer's injection, dextrose injection, dextrose+sodium chloride injection, PEG (polyethylene glycol), lactated Ringer's injection, ethanol, propylene glycol, non-volatile oils such as sesame oil, cottonseed oil, rapeseed oil, soybean oil, corn oil, ethyl oleate, isopropyl myristate, and benzyl benzoate; solubilizing agents such as sodium benzoate, sodium salicylate, sodium acetate, urea, urethane, monoethyl acetamide, butazolidine, propylene glycol, Tween, nicotinic acid amide, hexamine, dimethylacetamide; buffering agents such as weak acids and their salts (acetic acid and sodium acetate), weak bases and their salts (ammonia and ammonium acetate), organic compounds, proteins, albumin, peptones, gums; stabilizing agents such as sodium chloride, stabilizers such as sodium bisulfite (NaHSO3), carbon dioxide gas, sodium metabisulfite (Na2S2O5), sodium sulfite (Na2SO3), nitrogen gas (N2), ethylenediaminetetraacetic acid; antioxidants such as sodium bisulfite 0.1%, sodium formaldéhyde sulfoxylate, thiourea, ethylenediaminetetraacetic acid disodium, acetone sodium bisulfite; analgesics such as benzyl alcohol, chlorobutanol, procaine hydrochloride, glucose, calcium gluconate; and suspending agents such as CMC sodium, sodium alginate, Tween 80, and aluminum monostearate.

The suppository of the present disclosure may be used in bases such as cocoa butter, lanolin, Witepsol, polyethylene glycol, glycerogelatin, methylcellulose, carboxymethylcellulose, a mixture of stearic acid and oleic acid, Subanal, cottonseed oil, safflower oil, palm oil, cocoa butter+cholesterol, lecithin, lanette wax, monostearin glycerol, Tween or Span, Imhausen, Monolen (monostearic acid propylene glycol), glycerin, Adeps solidus, Buytyrum Tego-G, Cebes Pharma 16, Hexaride Base 95, Cotomar, Hydrokote SP, S-70-XXA, S-70-XX75 (S-70-XX95), Hydrokote 25, Hydrokote 711, Idropostal, Massa estrarium (A, AS, B, C, D, E, I, T), Massa-MF, Masupol, Masupol-15, Neosufostal-N, Paramount-B, Suposhiro (OSI, OSIX, A, B, C, D, H, L), Suppository Base IV Type (AB, B, A, BC, BBG, E, BGF, C, D, 299), Supostal (N, Es), Weco (W, R, S, M, Fs), and Tegester Triglyceride Base (TG-95, MA, 57), and similar substances.

The solid dosage forms for oral administration of the present disclosure may include tablets, powders, granules, and capsules, and such solid dosage forms are prepared by mixing the above-mentioned extract with at least one excipient, such as starch, calcium carbonate, sucrose, lactose, gelatin, and similar substances. Additionally, lubricants such as magnesium stearate and talc may be used, aside from simple excipients.

The liquid formulations for oral administration of the present disclosure may include suspensions, solutions, emulsions, syrups, and similar substances, and may also include various excipients, such as wetting agents, sweeteners, flavoring agents, preservatives, and similar substances, in addition to commonly used simple diluents such as water and liquid paraffin.

The formulations for non-oral administration include sterilized aqueous solutions, non-aqueous solvents, suspensions, emulsions, lyophilized formulations, and suppositories.

The non-aqueous solvents and suspending agents of the present disclosure may be used in propylene glycol, polyethylene glycol, vegetable oils such as olive oil, and injectable esters such as ethyl oleate, and similar substances.

The pharmaceutical composition of the present disclosure is administered in an amount effective for pharmacological activity.

In the present disclosure, the term “pharmaceutically effective amount” refers to an amount sufficient to treat the disease with a reasonable benefit/risk ratio applicable to medical treatment, and the effective dosage level can be determined based on factors such as the type and severity of the patient's condition, the activity of the drug, the patient's sensitivity to the drug, the administration time, the route of administration, the elimination rate, the treatment duration, other drugs used concomitantly, and other factors well-known in the medical field.

The pharmaceutically acceptable composition of the present disclosure may be administered as an individual therapeutic agent or in combination with other therapeutic agents, and may be administered sequentially or simultaneously with conventional therapeutic agents, either as a single or multiple doses.

It is important to administer an amount that provides the maximum effect with the minimum amount without side effects, considering all the aforementioned factors, and this can be easily determined by a person skilled in the art to which the present disclosure pertains.

The pharmaceutical composition of the present disclosure may be administered to an individual via various routes.

All methods of administration are foreseeable, including, for example, oral administration, subcutaneous injection, intraperitoneal administration, intravenous injection, intramuscular injection, intrathecal injection (epidural space), sublingual administration, buccal administration, rectal insertion, vaginal insertion, ocular administration, otic administration, nasal administration, inhalation, spraying through the mouth or nose, transdermal administration, and other forms of administration.

The pharmaceutical composition of the present disclosure is determined based on various relevant factors, including the disease to be treated, the administration route, the patient's age, gender, weight, severity of the disease, and the type of active ingredient.

In the present disclosure, the term “subject” refers to an individual in need of treatment for a disease, and more specifically includes mammals such as humans, non-human primates, mice, rats, dogs, cats, horses, and cattle.

In the present disclosure, the term “administration” refers to providing a predetermined composition of the present disclosure to a subject by any appropriate method.

In the present disclosure, the term “prevention” refers to any act of inhibiting or delaying the onset of a target disease, and the term “treatment” refers to any act by which the target disease and its associated metabolic abnormalities are improved or favorably altered by the administration of the pharmaceutical composition of the present disclosure. The term “improvement” refers to any act of reducing the severity of symptoms or other parameters related to the target disease through the administration of the composition of the present disclosure.

The present disclosure provides a method for screening a therapeutic agent for idiopathic pulmonary fibrosis, comprising:
(a) measuring the level of one ore more free fatty acid or arachidonic acid metabolite selected from the group comprising of myristic acid, heptadecanoic acid, 5(S)-HETE (5-hydroxyeicosatetraenoic acid), and 12(S)-HETE (12-hydroxyeicosatetraenoic acid) in exhaled breath condensate isolated from an idiopathic pulmonary fibrosis model treated with a candidate substance; and
(b) selecting the candidate substance as a therapeutic agent for idiopathic pulmonary fibrosis if the level of one or more free fatty acid or arachidonic acid metabolite selected from the group comprising of myristic acid, heptadecanoic acid, 5(S)-HETE, and 12(S)-HETE is decreased in the exhaled breath condensate isolated from the subject.

Additionally, the present disclosure provides a method for screening a therapeutic agent for interstitial lung disease, comprising:
(a) measuring the level of one ore more free fatty acid or arachidonic acid metabolite selected from the group comprising of myristic acid, and 5(S)-HETE (5-hydroxyeicosatetraenoic acid) in exhaled breath condensate isolated from an interstitial lung disease model treated with a candidate substance; and
(b) selecting the candidate substance as a therapeutic agent for idiopathic pulmonary fibrosis if the level of one or more free fatty acid or arachidonic acid metabolite selected from the group comprising of myristic acid, heptadecanoic acid, 5(S)-HETE, and 12(S)-HETE is decreased in the exhaled breath condensate isolated from the subject.

In the present invention, the term “screening” refers to the process of selecting a substance with a specific property from a group of candidate substances through a particular manipulation or evaluation method.

Specifically, for the purposes of the present disclosure, the screening method involves a series of steps to assess the efficacy of candidate drug substances using the method described above, in order to identify the most effective therapeutic agent for idiopathic pulmonary fibrosis or interstitial lung disease, particularly for individuals with poor prognosis predicted to have solid tumors, although it is not limited to this.

The step of confirming the therapeutic response and effect can be repeated several times depending on the candidate substance, and additional substances or steps may be included for further confirmation of therapeutic response and effect. These steps are common in screening methods and can include additional procedures as conventionally used in the art, but this is not restrictive.

In the present invention, the term “candidate substance” refers to an unknown substance used in the screening process to measure the expression changes of the biomarker in the disease model of the invention. It can be selected from the group consisting of nucleotides, DNA, RNA, amino acids, aptamers, proteins, stem cells, stem cell culture media, compounds, microbial culture media or extracts, natural products, and natural extracts, but is not limited to these.

In the present invention, the term “treatment” refers to any action that improves or beneficially alters the condition of the targeted disease and its related metabolic abnormalities. This includes the use of chemotherapy, surgical procedures, biological therapy, and other methods.

In this context, if the levels of the biomarkers of the present invention in the substance being tested, compared to a control group (such as a normal control group), increase or decrease depending on the type of biomarker, it can be concluded that the targeted disease has been treated (or improved). As described above, “increased levels” refer to the previously mentioned criteria.

For the treatment of the targeted disease in the present invention, conventional therapeutic methods or conventional therapeutic drugs can be used, or the candidate substances disclosed in the present invention may be administered, but this is not restrictive.

In the present specification, the term “increased levels” refers to either the detection of a substance that was previously undetectable or an increase in the amount detected relative to normal levels.

For example, an “increase” in levels means that the level in the experimental group is at least 1%, 2%, 3%, 4%, 5%, 10%, or more, relative to the level in the control group. Specifically, it can mean an increase of 5%, 10%, 20%, 30%, 40%, 50%, 60%, 70%, 80%, 90%, or more, and/or an increase of 0.5-fold, 1.1-fold, 1.2-fold, 1.4-fold, 1.6-fold, 1.8-fold, or more.

More specifically, the increase may range from 1 to 1.5 times, 1.5 to 2 times, 2 to 2.5 times, 2.5 to 3 times, 3 to 3.5 times, 3.5 to 4 times, 4 to 4.5 times, 4.5 to 5 times, 5 to 5.5 times, 5.5 to 6 times, 6 to 6.5 times, 6.5 to 7 times, 7 to 7.5 times, 7.5 to 8 times, 8 to 8.5 times, 8.5 to 9 times, 9 to 9.5 times, 9.5 to 10 times, or 10 times or more compared to the control group. However, this is not restrictive.

The meaning of the opposing term can be understood by a person skilled in the art as being the opposite of the above definition, consistent with the opposite meaning.

The terms used in the present disclosure have been selected based on commonly used general terms currently prevalent, considering the functions within the context of the present disclosure. However, these terms may vary depending on the intent of those skilled in the art, case law, or the emergence of new technologies. Additionally, in certain cases, terms may be arbitrarily selected by the applicant, and in such cases, their meaning will be described in detail in the specification of the relevant invention. Therefore, the terms used in the present disclosure should be defined based not merely on the names of the terms themselves, but on their meanings and the content of the present disclosure as a whole.

In the specification of the present disclosure, when a certain part is said to “include” a particular component, it means that, unless explicitly stated otherwise, it is not excluding other components, but may include additional components. In the entire specification of the present disclosure, terms such as “about” and “substantially” are used to mean within the manufacturing and material tolerances inherent in the mentioned meaning, or close to the specified values, and are used to prevent unscrupulous infringers from unfairly exploiting the disclosed content where exact or absolute values are mentioned, in order to aid in the understanding of the present disclosure.

In the entire specification of the present disclosure, the term “combination thereof” included in the Markush-type expression refers to one or more mixtures or combinations selected from the group of components described in the Markush-type expression, meaning that it includes one or more selected from the group consisting of the components described above.

### Mode of the Invention

Hereinafter, preferred examples are provided to aid in understanding the present disclosure. However, the following examples are provided merely to facilitate understanding of the present disclosure and are not intended to limit the scope of the invention.

### EXAMPLE

### Example 1. Recruitment of Subjects and Comparison of Characteristics for Identification of Exhaled Biomarkers

To identify exhaled biomarkers for the diagnosis and prognosis of idiopathic pulmonary fibrosis, a group of idiopathic pulmonary fibrosis patients, a normal control group, and a disease control group were recruited. Specifically, 56 patients with idiopathic pulmonary fibrosis were recruited based on the criteria outlined in Table 1, and clinical data for this patient group were collected at the time of study enrollment according to Table 2. Additionally, a control group consisting of 59 individuals, including 31 normal controls and 28 disease controls, was recruited. The recruitment was conducted to ensure similarity in age and gender between the idiopathic pulmonary fibrosis patient group and the control group, while the disease control group consisted of patients with interstitial lung disease (non-IPF ILD) other than idiopathic pulmonary fibrosis.

TABLE 1

| Classification | Description |
|---|---|
| Registration | \textcircled{1} Shows findings of Usual Interstitial Pneumonia (UIP) |
| Criteria | on chest CT |
| | \textcircled{2} Or, UIP pattern confirmed by surgical lung biopsy |
| Exclusion | \textcircled{1} In cases with known causes that can induce interstitial |
| Criteria | lung disease (such as environmental exposure at |
| | residence or workplace, connective tissue disease, or |
| | drug toxicity) |
| | \textcircled{2} In cases with severe conditions that may affect |
| | pulmonary function tests (e.g., lung resection, destroyed |
| | lung due to tuberculosis, bronchiectasis, pulmonary |
| | hypertension, etc.). |

TABLE 2

| Classification | Description |
|---|---|
| Personal | Age (Full Years: Difference Between Consent Year |
| Information | and Birth Year) |
| Written Consent | Clinical Research Consent Form and Biological |
| Form | Sample Consent Form |
| Medical History | Smoking history, past medical history |
| Chest imaging | Plain chest imaging and high-resolution CT (HRCT) |
| Pulmonary function | Forced vital capacity, diffusing capacity for carbon |
| | monoxide, lung volume (using methods |
| | recommended by the American Thoracic Society) |

The clinical characteristics at the time of study enrollment were compared among the recruited idiopathic pulmonary fibrosis patient group, normal control group, and disease control group. Table 3 presents a comparison of the clinical characteristics between the idiopathic pulmonary fibrosis patient group and the normal control group, and Table 4 presents a comparison of the clinical characteristics between the idiopathic pulmonary fibrosis patient group and the disease control group.

TABLE 3

| | Idiopathic | | |
|---|---|---|---|
| | Pulmonary Fibrosis | Normal control | |
| Classification | (IPF) patient group | group (Control) | p-value |
| Number of | 56 | 31 | |
| participants | | | |
| (persons) | | | |
| Age (years) | 68.5 | 62 | 0.001 |
| Gender ratio | 43:13 | 9:21 | <0.001 |
| (Male:Female) | | | |
| BMI (kg/m²) | 24.72 | 23.62 | 0.601 |
| (Average value) | | | |
| Smoking history | | | <0.001 |
| (persons (%)) | | | |
| No experience | 14(25) | 24(77.4) | |
| Stopped | 36(64.3) | 4(12.9) | |
| Currently smoking | 6(10.7) | 3(9.7) | |

TABLE 4

| | Idiopathic | | |
|---|---|---|---|
| | Pulmonary Fibrosis | Non-IPF, ILD | |
| Classification | (IPF) patient group | control group | p-value |
| Number of | 56 | 28 | |
| participants | | | |
| (persons) | | | |
| Age (years) | 68.5 | 69.5 | 0.497 |
| Gender ratio | 43:13 | 23:5 | 0.573 |
| (Male:Female) | | | |
| BMI (kg/m²) | 24.72 | 25.16 | 0.249 |
| (Average value) | | | |
| Smoking history | | | 0.563 |
| (persons (%)) | | | |
| No experience | 14(25) | 6(21.4) | |
| Stopped | 36(64.3) | 22(78.6) | |
| Currently smoking | 6(10.7) | 0 | |
| FVC(% predicted) | 74.5 | 73.5 | 0.465 |

The comparison of clinical characteristics revealed that the idiopathic pulmonary fibrosis patient group was older than the normal control group (68.5 years vs. 62 years), had a higher proportion of males (77% vs. 29%), and included more smokers (75% vs. 23%). However, no differences were observed between the idiopathic pulmonary fibrosis patient group and the disease control group in terms of age, sex, smoking history, or lung function.

### Example 2. Discovery of Exhaled Biomarkers

### Example 2-1. Collection of Exhaled Gases

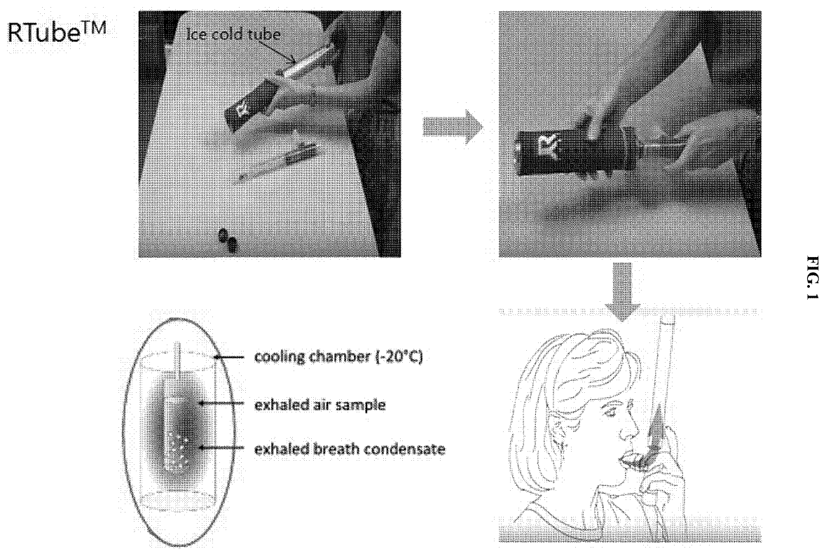

Exhaled breath was collected from the IPF patient group, normal control group, and disease control group recruited according to Example 1. Specifically, exhaled breath condensate (EBC) was collected using a condenser. Exhaled breath condensate is defined as the exhaled air that is cooled upon contact with the cooled surface of the condenser. The cooled air, which is either in liquid form or frozen, was immediately analyzed or stored for analysis after a certain period. It was classified into volatile or non-volatile polymer substances. In this example, exhaled breath condensate was collected using a condenser (RTube_exhaled breath condensate collector, Respiratory Research, Inc. part no. K001-A08) as shown in FIG. 1.

### Example 2-2. Method for Identifying Disease-Specific Metabolites in Exhaled Breath

To identify disease-specific metabolites in exhaled breath, free fatty acids in the collected breath were analyzed. To this end, the analytical substances were concentrated and then analyzed using GC-MS (Gas Chromatography-Mass Spectrometry) and SPME (Solid Phase Micro-Extraction) techniques. Specifically, a fiber coated with a material capable of adsorbing organic compounds was placed into a container with exhaled breath gas to adsorb the organic compounds. This fiber was then placed into the injector of the GC-MS, where the adsorbed organic compounds were desorbed at high temperature, allowing the organic compounds to flow into the analytical device for analysis.

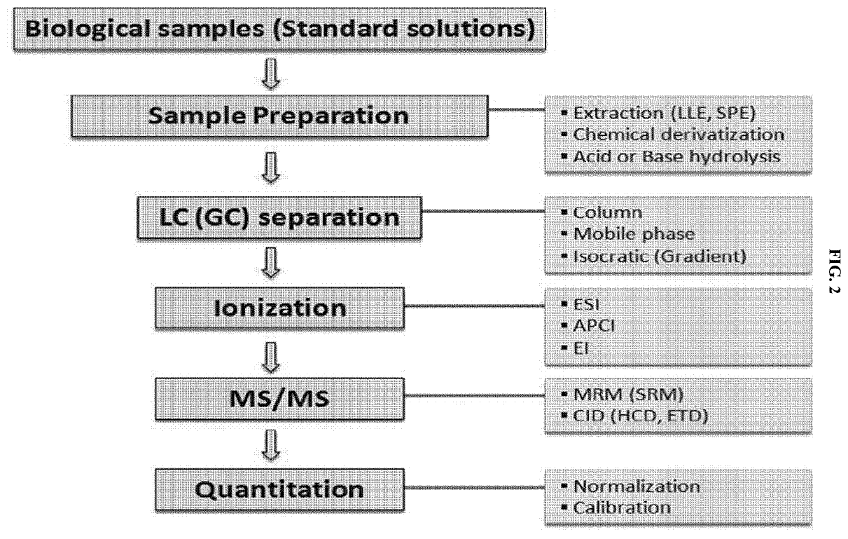

The analysis of the exhaled breath condensate was performed using the same method as the metabolite analysis in standard blood samples. In the present disclosure, free fatty acids in the exhaled breath were extracted with an organic solvent, followed by derivatization through a chemical reaction, and analyzed by GC-MS. Eicosanoids, which are arachidonic acid metabolites, were extracted using a solid-phase cartridge and analyzed using LC-MS/MS [MRM] (Liquid Chromatography-Mass Spectrometry with multiple reaction monitoring) (FIG. 2).

### Example 2-3. Results of Identification of Disease-Specific Metabolites in Exhaled Breath

In Example 1, exhaled breath condensate of 1 ml per subject was collected from 56 IPF patients, 31 normal controls, and 28 disease controls according to the method of Example 2-1. From this, disease-specific metabolites for idiopathic pulmonary fibrosis were identified using the method of Example 2-2.

As a result of analyzing the metabolites by condensing the exhaled breath collected from the patients, five types of free fatty acids were detected (Table 5), and six types of arachidonic acid metabolites (eicosanoids) were identified (Table 6).

TABLE 5

| Classification | Free fatty acids |
|---|---|
| 1 | Myristic acid (C14:0) |
| 2 | Pentadecanoic acid (C15:0) |
| 3 | Palmitic acid (C16:0) |
| 4 | Heptadecanoic acid (C17:0) |
| 5 | Stearic acid (18:0) |

TABLE 6

| Classification | Eicosanoids |
|---|---|
| 1 | LTB4 |
| 2 | 5(S)-HETE |
| 3 | 12(S)-HETE |
| 4 | 17(S)-DiHDoHE |
| 5 | 11,12-EET |
| 6 | 8(9)-DHET |

Additionally, the levels of the disease-specific metabolites in the exhaled

breath of the IPF patient group were compared with those of the normal control group and disease control group, and the data were presented as medians (interquartile range) or numbers (%).

First, the comparison of metabolites in the exhaled breath between the IPF patient group and the normal control group (Table 7) showed that among the five free fatty acids, myristic acid was present at significantly higher concentrations in the IPF group compared to the normal control group (IPF vs. control, p=0.02). Additionally, among the six arachidonic acid metabolites, 5(S)-HETE and 12(S)-HETE were found at significantly higher concentrations in the IPF group compared to the normal control group (5(S)-HETE: p<0.001, 12(S)-HETE: p=0.006).

TABLE 7

| Classification | IPF | Control | P value |
|---|---|---|---|
| Myristic acid | 0.001125 | 0.001 | 0.019 |
| (ug/ul) | (0.0009925, 0.00119) | (0.00097, 0.00115) | |
| Pentadecanoic acid | 0.000405 | 0.000415 | 0.604 |
| (ug/ul) | (0.00036125, 0.000505) | (0.00035, 0.0004827590) | |
| Palmitic acid | 0.0726525 | 0.076125 | 0.264 |
| (ug/ul) | (0.0605475, 0.0909) | (0.05769, 0.08166) | |
| Heptadecanoic acid | 0.0006425 | 0.000615 | 0.302 |
| (ug/ul) | (0.0005075, 0.0007625) | (0.00055, 0.0007) | |
| Stearic acid | 0.035075 | 0.03206 | 0.208 |
| (fmol/ul) | (0.0301425, 0.050125) | (0.02523, 0.04435) | |
| LTB4 | 0.4414116500(0.3005723, | 0.4409841075(0.3439329745, | 0.608 |
| (fmol/ul) | 0.5999440410) | 0.6564423730) | |
| (S)5-HETE | 0.0914444250(0.0493554210, | 0.03272694160 | <0.001 |
| (fmol/ul) | 0.2256412500) | (0, 0.0674662170) | |
| (S)12-HETE | 0 | 0 | 0.006 |
| (fmol/ul) | (0, 0.1187410990) | (0, 0) | |
| 0(S),17(S)- | 0.2122121190(0.1448132900, | 0.2342839145(0.1355930998, | 0.758 |
| DiHDoHE(fmol/ul) | 0.3109492310) | 0.3193676427) | |
| 11,12-EET | 0.7641422200(0.3771398110, | 0.7448633260(0.1868594940, | 0.81 |
| (fmol/ul) | 1.282605749) | 1.327662976) | |
| 8(9)-DHET | 0.8852249300(0.6697271970, | 0.9682439785(0.7502522090, | 0.58 |
| (fmol/ul) | 1.386258516) | 1.163670803) | |

Additionally, the comparison of metabolites in the exhaled breath between the IPF patient group and the disease control group (Table 8) showed that among the five free fatty acids, myristic acid was present at significantly higher concentrations in the IPF group compared to the disease control group (IPF vs non-IPF, p=0.05). Furthermore, among the six arachidonic acid metabolites, 5(S)-HETE and 12(S)-HETE were found at significantly higher concentrations in the IPF group compared to the disease control group (5(S)-HETE: p=0.004, 12(S)-HETE: p=0.004).

TABLE 8

| Classification | IPF | non-IPF | p value |
|---|---|---|---|
| Myristic acid | 0.001125 | 0.000985 | 0.054 |
| (ug/ul) | (0.0009925, 0.00119) | (0.0009325, 0.001195) | |
| Pentadecanoic acid | 0.000405 | 0.00036 | 0.074 |
| (ug/ul) | (0.00036125, 0.000505) | (0.00032625, | |
| | | 0.00043375) | |
| Palmitic acid | 0.0726525 | 0.0673425 | 0.147 |
| (ug/ul) | (0.0605475, 0.0909) | (0.05880375, | |
| | | 0.08035875) | |
| Heptadecanoic acid | 0.0006425 | 0.000565 | 0.048 |
| (ug/ul) | (0.0005075, 0.0007625) | (0.000505, 0.00064875) | |
| Stearic acid | 0.035075 | 0.035485 | 0.448 |
| (fmol/ul) | (0.0301425, 0.050125) | (0.027695, 0.046105) | |
| LTB4 | 0.4414116500 | 0.3706585020 | 0.321 |
| (fmol/ul) | (0.3005723, | (0.2478913770, | |
| | 0.5999440410) | 0.515180235) | |
| (S)5-HETE | 0.0914444250 | 0.0354203270 | 0.004 |
| (fmol/ul) | (0.0493554210, | (0, 0.0993325070) | |
| | 0.2256412500) | | |
| (S)12-HETE | 0 | 0 | 0.019 |
| (fmol/ul) | (0, 0.1187410990) | (0, 0) | |
| 0(S),17(S)- | 0.2122121190 | 0.2269089630 | 0.589 |
| DiHDoHE(fmol/ul) | (0.1448132900, | (0.1703996680, | |
| | 0.3109492310) | 0.3241363110) | |
| 11,12-EET | 0.7641422200 | 0.6198018830 | 0.875 |
| (fmol/ul) | (0.3771398110, | (0.2691207370, | |
| | 1.282605749) | 1.481593330) | |
| 8(9)-DHET | 0.8852249300 | 0.9609862320 | 0.603 |
| (fmol/ul) | (0.6697271970, | (0.6204930930, | |
| | 1.386258516) | 1.241917583) | |

### Example 3. Clinical Validation of the Identified Exhaled Biomarkers

### Example 3-1. Clinical Analysis Method for Exhaled Biomarkers

The exhaled metabolites identified according to Example 2 were examined for their correlation with disease diagnosis, disease progression, and acute exacerbation.

Specifically, disease diagnosis was confirmed by performing ROC analysis of exhaled metabolites in samples collected from the IPF patient group, ILD patient group, and normal control group.

Disease progression was defined as a decrease of more than 10% in forced vital capacity (FVC) over the 6 months following the clinical sample collection, and the percentage was calculated according to the following formula.

Forced Vital Capacity (FVC) decrease (%)=(FVC at the observed time point−FVC at the collection time point)/FVC at the collection time point×100

Finally, acute exacerbation was defined with reference to the 2007 definition by the American Thoracic Society (Table 9), and the relationship with clinical indicators was assessed using regression models and receiver operating characteristic (ROC) analysis. Sensitivity, specificity, accuracy, and correlations with pulmonary function (forced vital capacity, diffusing capacity for carbon monoxide, lung volume) and exercise capacity (6-minute walk test distance, minimum oxygen saturation) at the time of sample collection were confirmed.

TABLE 9

| Classification | Definition of acute exacerbation |
|---|---|
| 1 | Previously diagnosed with idiopathic pulmonary fibrosis |
| | (IPF) |
| 2 | Recently occured or worsened shortness of breath within |
| | the last month |
| 3 | New ground-glass opacity or lesions observed on chest |
| | CT |
| 4 | No pulmonary infection detected in bronchial aspirate |
| | or bronchoalveolar lavage fluid |
| 5 | Exclusion of known causes that can induce left heart |
| | failure, pulmonary embolism, or acute lung injury |

### Example 3-2. Evaluation of Diagnostic Ability for Idiopathic Pulmonary Fibrosis

The diagnostic predictive ability of five free fatty acids and six eicosanoids detected in the patients' exhaled breath condensate, as identified in Example 2, for idiopathic pulmonary fibrosis (IPF) was evaluated through receiver operating characteristic (ROC) curve analysis. Specifically, ROC curve analysis was performed to assess the predictive value of exhaled biomarkers for survival, and survival prediction was evaluated from the sampling date using Kaplan-Meier survival analysis and the log-rank test. Independent risk factors for mortality were identified through Cox proportional hazards analysis. All significance tests were two-tailed, and p-values less than 0.05 were considered indicative of statistical significance. The analysis was performed using SPSS statistics (version 24.0; IBM Corp., Armonk, NY, USA).

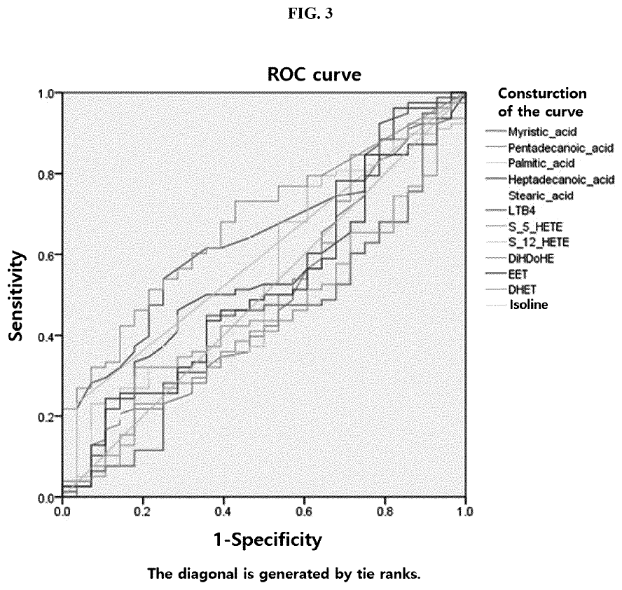

As a result, among the free fatty acids, myristic acid, and among the arachidonic acid metabolites, 5(S)-HETE and 12(S)-HETE were identified as significant diagnostic markers for IPF (FIG. 3). Specifically, the AUC (Area under the ROC curve) was found to be between 0.635 and 0.756, indicating a high value close to 1. The p-value for myristic acid was 0.003, for 5(S)-HETE was <0.001, and for 12(S)-HETE was 0.049 (Table 10).

TABLE 10

| Classification | Area | p-value | lower 95% CI | upper 95% CI |
|---|---|---|---|---|
| Myristic acid | 0.705 | 0.003 | 0.586 | 0.825 |
| (ug/ul) | | | | |
| Pentadecanoic acid | 0.519 | 0.786 | 0.381 | 0.656 |
| (ug/ul) | | | | |
| Palmitic acid | 0.565 | 0.341 | 0.432 | 0.698 |
| (ug/ul) | | | | |
| Heptadecanoic acid | 0.604 | 0.128 | 0.474 | 0.734 |
| (ug/ul) | | | | |
| Stearic acid | 0.606 | 0.122 | 0.475 | 0.737 |
| (fmol/ul) | | | | |
| LTB4 | 0.465 | 0.608 | 0.331 | 0.599 |
| (fmol/ul) | | | | |
| 5(S)-HETE | 0.756 | <0.001 | 0.649 | 0.864 |
| (fmol/ul) | | | | |
| 12(S)-HETE | 0.635 | 0.049 | 0.512 | 0.757 |
| (fmol/ul) | | | | |
| 0(S),17(S)- | 0.479 | 0.758 | 0.344 | 0.614 |
| DiHDoHE(fmol/ul) | | | | |
| 11,12-EET | 0.516 | 0.81 | 0.383 | 0.65 |
| (fmol/ul) | | | | |
| 8(9)-DHET | 0.462 | 0.58 | 0.331 | 0.593 |
| (fmol/ul) | | | | |

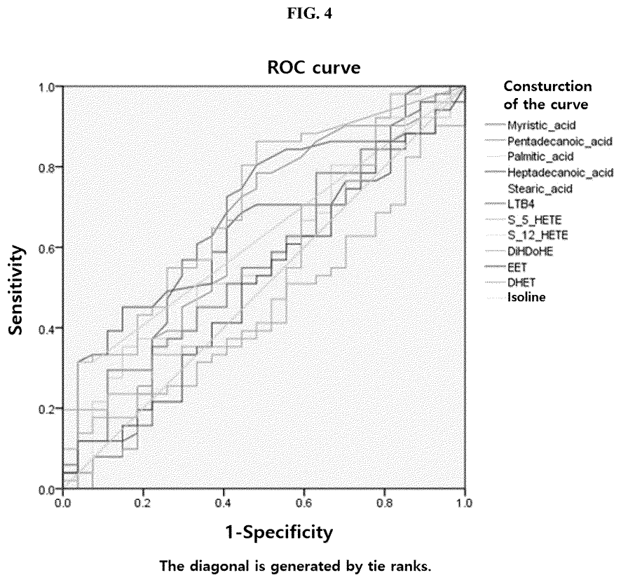

Additionally, as a result of comparing the IPF patient group with the disease control group, it was confirmed that heptadecanoic acid among the free fatty acids and 5(S)-HETE among the arachidonic acid metabolites functioned as effective diagnostic markers for distinguishing IPF from interstitial lung disease (FIG. 4). Specifically, the AUC was found to range from 0.642 to 0.699, with a p-value of 0.04 for heptadecanoic acid and a p-value of 0.004 for 5(S)-HETE (Table 11).

TABLE 11

| Classification | Area | p-value | lower 95% CI | upper 95% CI |
|---|---|---|---|---|
| Myristic acid | 0.633 | 0.054 | 0.493 | 0.774 |
| (ug/ul) | | | | |
| Pentadecanoic acid | 0.612 | 0.105 | 0.471 | 0.754 |
| (ug/ul) | | | | |
| Palmitic acid | 0.58 | 0.25 | 0.447 | 0.712 |
| (ug/ul) | | | | |
| Heptadecanoic acid | 0.642 | 0.04 | 0.518 | 0.767 |
| (ug/ul) | | | | |
| Stearic acid | 0.543 | 0.532 | 0.409 | 0.677 |
| (fmol/ul) | | | | |
| LTB4 | 0.569 | 0.321 | 0.436 | 0.701 |
| (fmol/ul) | | | | |
| (S)5-HETE | 0.699 | 0.004 | 0.576 | 0.823 |
| (fmol/ul) | | | | |
| (S)12-HETE | 0.62 | 0.083 | 0.494 | 0.746 |
| (fmol/ul) | | | | |
| 0(S),17(S)- | 0.463 | 0.589 | 0.333 | 0.592 |
| DiHDoHE(fmol/ul) | | | | |
| 11,12-EET | 0.511 | 0.875 | 0.375 | 0.647 |
| (fmol/ul) | | | | |
| 8(9)-DHET | 0.536 | 0.603 | 0.398 | 0.674 |
| (fmol/ul) | | | | |

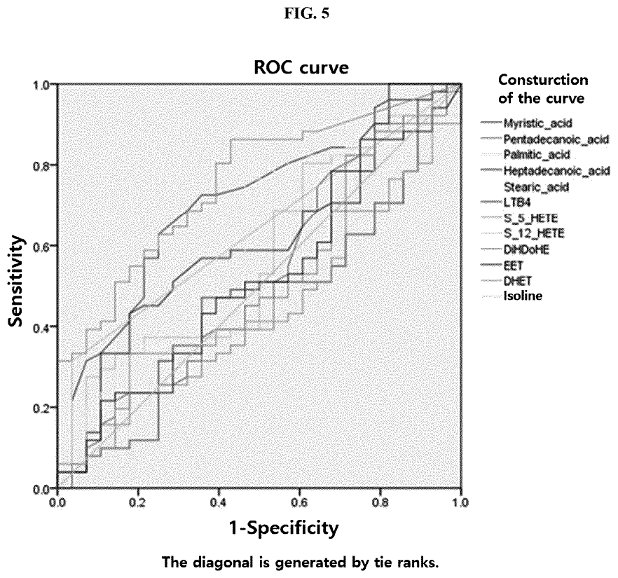

Finally, when comparing the disease control group with the normal control group, myristic acid among the free fatty acids and 5(S)-HETE among the arachidonic acid metabolites were found to be effective diagnostic markers for interstitial lung disease (FIG. 5). Specifically, the AUC ranged from 0.642 to 0.680, with a p-value of 0.026 for myristic acid and a p-value of 0.005 for 5(S)-HETE (Table 12).

TABLE 12

| Classification | Area | p-value | lower 95% CI | upper 95% CI |
|---|---|---|---|---|
| Myristic acid | 0.642 | 0.026 | 0.528 | 0.757 |
| (ug/ul) | | | | |
| Pentadecanoic acid | 0.485 | 0.819 | 0.358 | 0.612 |
| (ug/ul) | | | | |
| Palmitic acid | 0.537 | 0.562 | 0.412 | 0.663 |
| (ug/ul) | | | | |
| Heptadecanoic acid | 0.551 | 0.426 | 0.428 | 0.674 |
| (ug/ul) | | | | |
| Stearic acid | 0.592 | 0.152 | 0.469 | 0.714 |
| (fmol/ul) | | | | |
| LTB4 | 0.442 | 0.367 | 0.319 | 0.566 |
| (fmol/ul) | | | | |
| 5(S)-HETE | 0.680 | 0.005 | 0.573 | 0.787 |
| (fmol/ul) | | | | |
| 12(S)-HETE | 0.595 | 0.138 | 0.480 | 0.710 |
| (fmol/ul) | | | | |
| 0(S),17(S)- | 0.491 | 0.886 | 0.362 | 0.620 |
| DiHDoHE(fmol/ul) | | | | |
| 11,12-EET | 0.512 | 0.847 | 0.388 | 0.636 |
| (fmol/ul) | | | | |
| 8(9)-DHET | 0.457 | 0.505 | 0.338 | 0.577 |
| (fmol/ul) | | | | |

According to these results, it has been confirmed that exhaled metabolites can be effectively utilized for diagnosing idiopathic pulmonary fibrosis or interstitial lung disease by differentiating idiopathic pulmonary fibrosis from interstitial lung disease/normal control groups or interstitial lung disease from normal control group.

### Example 3-3. Verification of the Correlation between Exhaled Biomarkers and Pulmonary Function in Interstitial Lung Disease Patients

In Example 2, the correlation between the five free fatty acids and six arachidonic acid metabolites identified as biomarkers and pulmonary function in patients with interstitial lung disease was evaluated using Pearson's correlation coefficient. Specifically, pulmonary function was evaluated with respect to FVC (forced vital capacity), FEV1 (forced expiratory volume in 1 second), DLCO (diffusing capacity of the lung for CO), and TLC (total lung capacity).

As a result, all five detected free fatty acids showed a large absolute value of Pearson's correlation coefficient with respect to diffusing capacity of the lung for CO (DLCO), and the p-values were significantly low, indicating a statistically significant correlation. Additionally, among the six arachidonic acid metabolites, 11,12-EET showed a large absolute value of Pearson's correlation coefficient with total lung capacity (TLC) and forced vital capacity (FVC), and the p-value was low, indicating a statistically significant correlation (Table 13).

TABLE 13

| Classification | | | | |
|---|---|---|---|---|
| ( [symbol] ) | FVC(%) | FEV1(%) | DLCO(%) | TLC(%) |
| Myristic acid | −0.075 | −0.030 | −0.284 | −0.111 |
| | (p = 0.511) | (p = 0.791) | (p = 0.012) | (p = 0.333) |
| Pentadecanoic acid | −0.023 | −0.028 | −0.323 | −0.052 |
| | (p = 0.839) | (p = 0.805) | (p = 0.004) | (p = 0.652) |
| Palmitic acid | −0.089 | −0.112 | −0.351 | −0.117 |
| | (p = 0.434) | (p = 0.328) | (p = 0.002) | (p = 0.308) |
| Heptadecanoic aci | −0.131 | −0.134 | −0.258 | −0.144 |
| | (p = 0.248) | (p = 0.240) | (p = 0.023) | (p = 0.208) |
| Stearic acid | −0.196 | −0.184 | −0.364 | −0.208 |
| | (p = 0.084) | (p = 0.104) | (p = 0.001) | (p = 0.068) |
| LTB4 | 0.049 | 0.055 | 0.183 | 0.101 |
| (fmol/ul) | (p = 0.666) | (p = 0.633) | (p = 0.109) | (p = 0.377) |
| (S)5-HETE(fmol/ul) | −0.026 | −0.007 | 0.114 | 0.053 |
| | (p = 0.836) | (p = 0.959) | (p = 0.371) | (p = 0.678) |
| (S)12-HETE(fmol/ul) | −0.053 | 0.099 | 0.225 | 0.041 |
| | (p = 0.811) | (p = 0.653) | (p = 0.301) | (p = 0.851) |
| 0(S),17(S)- | 0.043 | −0.020 | 0.090 | 0.074 |
| DiHDoHE(fmol/ul) | (p = 0.706) | (p = 0.862) | (p = 0.439) | (p = 0.521) |
| 11,12-EET | −0.235 | −0.036 | −0.179 | −0.234 |
| | (p = 0.042) | (p = 0.757) | (p = 0.128) | (p = 0.045) |
| 8(9)-DHET(fmol/ul) | −0.050 | −0.083 | 0.114 | 0.023 |
| | (p = 0.663) | (p = 0.469) | (p = 0.319) | (p = 0.845) |

The description of the present disclosure above is for illustrative purposes, and those skilled in the art to which the present disclosure pertains will understand that the invention can be easily modified into other specific forms without altering the technical concept or essential features of the invention. Therefore, the embodiments described above should be understood as illustrative in all respects and not restrictive.

### INDUSTRIAL APPLICABILITY

The present disclosure relates to exhaled biomarkers for the diagnosis and prognosis patient with idiopathic pulmonary fibrosis patients. It involves selecting exhaled biomarkers from volatile organic compounds in exhaled gases, and the biomarkers according to the present disclosure are specific compounds for idiopathic pulmonary fibrosis. These biomarkers not only demonstrate excellent performance in distinguishing between idiopathic pulmonary fibrosis patients and both normal control and interstitial lung disease groups, but also enable diagnosis through a non-invasive method, making them useful for diagnosing and predicting the prognosis of idiopathic pulmonary fibrosis patients, including elderly patients, and thus have industrial applicability.

1-16. (canceled)

17. A method for detecting exhaled biomarkers and treating idiopathic pulmonary fibrosis, comprising:
(a) detecting a level of one or more exhaled biomarkers selected from the group consisting of myristic acid, heptadecanoic acid, 5(S)-HETE (5-Hydroxyeicosatetraenoic acid), and 12(S)-HETE (12-Hydroxyeicosatetraenoic acid) in exhaled breath collected from a subject; and
(b) treating the subject for idiopathic pulmonary fibrosis when the level of the one or more exhaled biomarker(s) selected from the group consisting of myristic acid, heptadecanoic acid, 5(S)-HETE, and 12(S)-HETE is increased compared to the corresponding exhaled biomarker level in a normal control group or an interstitial lung disease group;
wherein said treating comprises administering a therapeutically effective amount of a therapeutic agent.

18. The method of claim 17, wherein step (b), the subject:
is characterized by an increase in the level of one or more exhaled biomarkers, comprising myristic acid, 5(S)-HETE, or 12(S)-HETE, when compared to the corresponding level in a normal control group; or
is characterized by an increase in the level of one or more exhaled biomarkers, comprising heptadecanoic acid or 5(S)-HETE, when compared to the corresponding level in an interstitial lung disease (ILD) group.

19. The method of claim 17, wherein the exhaled biomarkers comprise one or more free fatty acids selected from the group consisting of myristic acid, pentadecanoic acid, palmitic acid, heptadecanoic acid, and stearic acid, and one or more arachidonic acid metabolites selected from the group consisting of LTB4, 5(S)-HETE, 12(S)-HETE, 17(S)-DiHDoHE3, 11,12-EET, and 8(9)-DHET,
wherein myristic acid, heptadecanoic acid, 5(S)-HETE, and 12(S)-HETE are selected based on the following criteria:
(i) when the control group is a normal control group, the p-value obtained from a t-test comparing the content of exhaled breath condensate between the subject and the normal control group is 0.019 or less for free fatty acids, and 0.006 or less for arachidonic acid metabolites; or
(ii) when the control group is an interstitial lung disease group, the p-value obtained from a t-test comparing the content of exhaled breath condensate between the subject and the interstitial lung disease group is 0.048 or less for free fatty acids, and less than 0.019 for arachidonic acid metabolites.

20. The method of claim 17, wherein the exhaled sample is exhaled breath condensate (EBC).

21. The method of claim 18, wherein, in the step (b), when compared to a normal control group, the AUC (area under the ROC curve) value for myristic acid, 5(S)-HETE, and 12(S)-HETE is greater than 0.63, or the p-value associated therewith is less than or equal to 0.049.

22. The method of claim 18, wherein, in the step (b), when compared to an interstitial lung disease group, the AUC value for heptadecanoic acid and 5(S)-HETE is greater than 0.64, or the p-value associated therewith is less than or equal to 0.04.

23. A method for detecting exhaled biomarkers and treating interstitial lung disease, comprising:
(a) detecting a level of one or more exhaled biomarkers selected from the group consisting of myristic acid and 5(S)-HETE (5-hydroxyeicosatetraenoic acid) in exhaled breath collected from a subject; and
(b) treating the subject for interstitial lung disease when the level of the one or more exhaled biomarkers selected from the group consisting of myristic acid and 5(S)-HETE is increased compared to the corresponding exhaled biomarker level in a normal control group;
wherein said treating comprises administering a therapeutically effective amount of a therapeutic agent.

24. The method of claim 23, wherein, the AUC value for myristic acid and 5(S)-HETE is greater than 0.6, or the p-value associated therewith is less than or equal to 0.026.

25. The method of claim 23, wherein the therapeutic agent comprises nintedanib, pirfenidone or other fibrotic drug.

26. The method of claim 23, wherein the therapeutic agent is prednisone or other corticosteroid.

27. The method of claim 23, wherein the therapeutic agent is cyclophosphamide, mycophenolate, azathioprine, or other immunosuppressant.
